(19) United States
(12) Patent Application Publication
(10) Pub. No.: US 2006/0147518 A1
(43) Pub. Date: Jul. 6, 2006

(54) Stable solid dispersion of a derivative of vinca alkaloid and process for manufacturing it

(75) Inventors: Joel Bougaret, Francarville (FR); Elie Leverd, Castres (FR); Marie-Dominique Ibarra, Souihanels (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: PIERRE FABRE MEDICAMENT

(21) Appl. No.: 11/025,348

(22) Filed: Dec. 30, 2004

(51) Int. Cl. A61K 31/4745; A61K 9/64; A61K 9/14

(52) U.S. Cl. 424/456; 514/283; 424/486

(57) ABSTRACT

This invention relates to solid and stable dispersions of a hydrosoluble derivative of vinca alkaloids in at least one polyethyleneglycol with a molecular mass between 800 and 30 000.

This invention relates to solid and stable dispersions of hydrosoluble derivatives of vinca alkaloids and more particularly derivatives of vinorelbine, particularly vinorelbine ditartrate in at least one polyethyleneglycol, which are intended to be incorporated into pharmaceutical compositions for oral administration of such a vinca derivative.

Antineoplastic chemotherapy was initially developed using intravenous methods. The arguments in favor of this administration method are:

lesser gastrointestinal toxicity,

total bioavailability, and

potentially lower inter and intra patient exposure variations than with an oral method.

However, the intravenous method is associated with serious disadvantages that limit its use: the morbidity of vein access, possible complications of central vein channels (infection, thrombosis), the risk of extravasation.

For several years, oral forms of antineoplastic chemotherapy have developed increasingly due to the real benefit possible for the patient. Furthermore, pharmaco-economic considerations that are becoming increasingly important in the choice of therapeutic strategies, are also leading towards the development of oral treatments.

A lot of exploratory work has been carried out on the possible use of molecules intended for the treatment of cancer and administrated by mouth, for former active principles (for example etoposide, cyclophosphamide and idarubicine), new synthetic derivatives of fluoropyridines (for example UFT, capecitabine, S-1), derivatives of platinum (for example JM-216) or Vinca alkaloids (e.g. vinorelbine).

Therefore this invention also concerns stable pharmaceutical compositions for oral administration of vinca alkaloids, and particularly vinorelbine in dispersed form.

Vinorelbine or 3′4′-Didehydro-4′-desoxy-8′-norvincaleucoblastine is an alkaloid derivative of vinca which exerts a cytostatic effect by inhibition of the polymerization of tubulin.

Vinorelbine, and more particularly a salt of vinorelbine, vinorelbine ditartrate, is also active in the treatment of large cell lung cancer and breast cancer. An injectable form was marketed for the first time in France in 1989. It is now marketed throughout the world in the form of a solution to be diluted for perfusion, to a concentration of 10 mg/ml expressed in basic vinorelbine and distributed in flasks with unit volumes of 1 and 5 ml.

More recently, an oral formulation of vinorelbine in solution was developed and put on the market under the name of NAVELBINE Oral® soft capsules. It is in the form of a soft gelatin capsule containing vinorelbine ditartrate and an excipient mix comprising polyethyleneglycol, glycerol, ethanol and water. The average molecular mass of polyethyleneglycol is between 200 and 600: these are liquid polyethyleneglycols such as MACROGOL 400. Unit doses expressed in basic vinorelbine are between 5 mg and 100 mg, and more advantageously equal to 20 mg, 30 mg, 40 mg and 80 mg.

These soft capsules were described in a patent application R.P. Scherer Technologies, Inc. WO 03/101383.

Pharmaceutical compositions according to this invention are intended for oral administration of alkaloid derivatives of vinca and particularly vinorelbine, in dispersed form. They contain the hydrosoluble derivative of vinca alkaloid, advantageously a salt of vinorelbine, and more particularly ditartrate dispersed in semi-solid or solid polyethyleneglycols.

More precisely, the stable solid dispersion according to the invention is associated with a hydrosoluble derivative of vinca alkaloid, particularly in at least one polyethyleneglycol with a molecular mass between 800 and 30 000, and more particularly a polyethyleneglycol with a molecular mass of between 1 000 and 6 000.

Polyethyleneglycols chosen in the invention have an average molecular mass greater than about 800. When the molecular mass is between 800 and 2 000, they are in semi-solid form, and when the molecular mass is higher, they are in solid form. They are differentiated from each other by their melting point, as indicated in the table below.

| | Polyethyleneglycol | | | | | | |
|---|---|---|---|---|---|---|---|
| | (average molecular mass) | | | | | | |
| | 1000 | 1500 | 4000 | 6000 | 8000 | 20000 | 30000 |
| Melting point | 37° C. | 44° C. | 50° C. | 55° C. | 60° C. | 60° C. | 65° C. |
| | to | to | to | to | to | to | to |
| | 40° C. | 48° C. | 58° C. | 63° C. | 63° C. | 63° C. | 70° C. |

According to one advantageous embodiment of this invention, the ratio of the masses of the hydrosoluble derivative of vinca alkaloids and more particularly firstly vinorelbine ditartrate, and secondly polyethyieneglycol, is between 1.5:1 and 1:10 and preferably between 1:3 and 1:6.

These dispersions of the derivative of vinca alkaloids or the salt of vinorelbine in polyethyleneglycols according to this invention form a solid dispersion. In general, the use of the solid dispersions technology in the pharmaceutical formulation domain is known. The first reason for the development of solid dispersions is based on the possibility of improving dissolution and therefore potentially the bioavailability of active principles that are not very soluble in water and are administered by mouth.

The use of hydrophilic polymers such as polyethyleneglycols, polyvinylpyrrolidone or cellulose derivatives tends towards this hydrosolubilisation. Within the context of this invention, solid dispersions are not used with the intention of increasing the dissolution rate of active constituents. Hydrosoluble derivatives of vinca alkaloids and particularly vinorelbine salts, and more particularly ditartrate, are very soluble in water and their wettability characteristics do not cause any problem.

However, unexpectedly, galenic forms of hydrosoluble derivatives of vinca alkaloids and particularly vinorelbine salts according to this invention are more stable.

Thus, vinorelbine ditartrate must be kept at a temperature of below −15° C., regardless of its form (amorphous or crystalline) and its degree of division (unground, ground or micronized).

On the other hand, solutions of vinorelbine ditartrate can be kept at temperatures between +50° C. and ±3° C. This is the case both for the injectable water based solution for injectable preparations, and for the soft capsules filling solution composed of liquid polyethyleneglycol, glycerol, ethanol and water. Therefore, it appeared that the solubilization operation was responsible for better stability.

Surprisingly, in the pharmaceutical compositions according to this invention, hydrosoluble derivatives of vinca alkaloids and particularly vinorelbine ditartrate which is in the dispersed powder state, are at least as stable, or even more stable, than the soft capsules in which they are dissolved.

A preparation of dispersions of hydrosoluble derivatives of vinca alkaloid, and particularly vinorelbine, and more particularly vinorelbine ditartrate, always begins with a mix of this active principle with polyethyleneglycol in the molten state. To achieve this, the said polyethyleneglycol will be previously heated to a temperature slightly greater than its melting temperature to bring it into the liquid state so that it can be mixed with the hydrosoluble derivative of vinca alkaloid while stirring. The process terminates with a cooling operation of the said dispersion to bring it into the solid state. If a polyethyleneglycol with a high molar mass is used, it will preferably be heated in the presence of a plastifier, which will bring the said solid polyethylene into the liquid state without exceeding a temperature of the order of 80° C.

The first step in the preparation of the solid dispersion can advantageously be done as follows:

either discontinuously: manufacturing in tank, before distribution of the mix for example in hard gelatin capsules or by the use of techniques such as molding injection,

or continuously using hot extrusion techniques.

These techniques have two advantages:

the concentration of the active principle in the final mix can be as high as 60%, which for example allows large unit doses,

the residence time of the active principle in the extruder, for which the duration of its exposure to high temperatures is short so that a vinorelbine salt can be used although it is sensitive to heat with polyethyleneglycols with a high molecular mass.

The dispersions obtained may be in divided form, for example in the form of pellets, or in monolithic form, for example in the form of tablets. In order to protect manufacturing personnel or the patient from risks of exposure to cytotoxic vinorelbine salts, the final pharmaceutical forms will be distributed in hard gelatin capsules or they will be coated tablets.

After mixing and cooling, polyethyleneglycol and vinorelbine give a mass that can be treated differently as a function of the particular form searched for. It may be directly poured into the hard gelatin capsules to lead to a monolithic form after the said hard gelatin capsules have been cooled.

Traditionally, the hard gelatin capsules are composed of gelatin, hydroxypropylmethycellulose or extracellular bacterial polysaccharide obtained using Aureobasidium pullulans, known under the name of pullulan.

According to one variant of the process according to this invention, the stable solid dispersion is extruded to obtain pellets to be used to make hard gelatin capsules or tablets. In the latter case, coating is done during the actual manufacturing operation, for example using a co-extruded technique, the dispersion being effectively co-extruded with a natural or synthetic film-forming polymer to obtain film-coated tablets directly.

As a variant, this type of coating operation may also be done during a later additional manufacturing step, for example requiring fluidized air bed or turbine coating techniques.

In both coating variants, the coating may advantageously be obtained using a film forming polymer, with natural or synthetic origin, and particularly cellulose derivatives such as hydroxypropylmethylcellulose, hydroxypropylcellulose or acrylic ester or modified methacrylic ester copolymers or polyethyleneglycols with high molecular weight.

When the said stable solid dispersion uses polyethyleneglycols with low molecular mass (800-2 000), technical additives such as structuring agents, and particularly silica, polyethylene oxide, microcrystalline cellulose, can be added. The proportions in which these additional structuring agents will be present vary between 0.05% and 10%, and preferably between 0.5% and 5%.

Finally, it should be noted that when polyethyleneglycols with a high molecular mass are used, it may be advantageous to add plastifiers to avoid an excessive increase in the melting temperature so that they can be obtained in the liquid state in the context of the first mixing operation with vinca derivatives. Examples of plastifiers include ester citrates, triacetine, etc.

The following examples describe some possible formulations and preparation processes:

### EXAMPLE 1

The use of a semi-solid polyethyleneglycol involves the incorporation of a structuring agent such as silica, as described in the following composition:

| | | |
|---|---|---|
| Vinorelbine ditartrate (in amorphous form) | 55.40 | mg |
| i.e. vinorelbine | 40.00 | mg |
| Silica | 3.00 | mg |
| Polyethyleneglycol 1000 | qsq 330.00 | mg |

The preparation is made discontinuously using a preliminary hot mix in a tank before distribution in gelatin capsules.

### EXAMPLE 2

The use of a solid polyethyleneglycol with a high melting point imposes the use of a plastifier and the use of a hot extrusion manufacturing process.

The following hot mix was prepared continuously in a co-extruder with a double screw:

| | | |
|---|---|---|
| Vinorelbine ditartrate (in amorphous form) | 55.40 | mg |
| i.e. vinorelbine | 40.00 | mg |
| Triethyl citrate | 6.00 | mg |
| Polyethyleneglycol 6000 | qsq 150.00 | mg |

Example 3 below gives a complete illustration of this invention and describes a process of production. It relates to a gelatin capsule containing 40 mg of vinorelbine dispersed in polyethyleneglycol 1500.

### EXAMPLE 3

The exact composition of the contents is:

| | | |
|---|---|---|
| Vinorelbine ditartrate (in amorphous form) | 55.40 | mg |
| i.e. vinorelbine | 40.00 | mg |
| Polyethyleneglycol 1500 | qsq 330.00 | mg |
| Size 2 gelatin capsule | 1 | |

The manufacturing process includes the following steps:

polyethyleneglycol 1500 is heated to a temperature of between 55° C. and 60° C.,

dispersion under mechanical stirring of vinorelbine ditartrate,

filling in size 2 hard gelatin capsules, with 330 mg of mix per hard gelatin capsule,

cooling to ambient temperature.

The essential constituent of the gelatin capsule casing is a hydrophilic polymer which, as mentioned above, may be gelatin or hydroxypropylmethylcellulose (HPMC) or pullulan.

There is no need to seal the gelatin capsules since no leakage occurs during storage. However, with regard to the cytotoxicity of the v-norelbine, it is recommended that it should be sealed for safety reasons. This is done either by stretch wrapping, or by spraying with a hydro-alcohol spray.

This composition has an excellent physicochemical stability: degradation of the dispersed vinorelbine formulated in hard gelatin capsules after 6 months of storage at 25° C./60% RH (severe temperature condition) is:

very significantly less than the degradation observed for vinorelbine alone,

less than or equal to the degradation observed in soft capsules.

The results are expressed below:

| | Vinorel- | Formulated vinorelbine | | |
|---|---|---|---|---|
| | bine | Hard | | |
| | ditar- | gelatin | HPMC | Soft |
| | trate | capsule | capsule | capsule |
| Physical state of | Powder | Dispersed | Dispersed | Solution |
| vinorelbine ditartrate | | powder | powder | |
| Total impurities | +1.87 | +0.70 | +0.62 | +0.76 |
| Including identified | | | | |
| impurities: | | | | |
| S/D6 | +1.02 | — | — | — |
| 6′-N-oxyvinorelbine | +0.37 | — | — | — |
| 6′-N-methylvinorelbine | +0.05 | +0.10 | +0.10 | +0.15 |
| 4-O-deacetylvinorelbine | +0.05 | +0.04 | +0.08 | +0.37 |
| 23-O-demethylvinorelbine | — | +0.15 | +0.17 | +0.16 |

Variation of the Content of Impurities (in % Relative) after 6 Months 25° C./60% RH Compared with t0.

Other hydrophilic polymers such as polyethyleneglycols were tested. The stability of vinorelbine in the presence of these other polymers is significantly lower: after only 1 month at 25° C./60% RH, the variation of the content of impurities compared with t0 was +7.63% and +29.08% for polyvinylpyrrolidone and a cellulosic ether respectively.

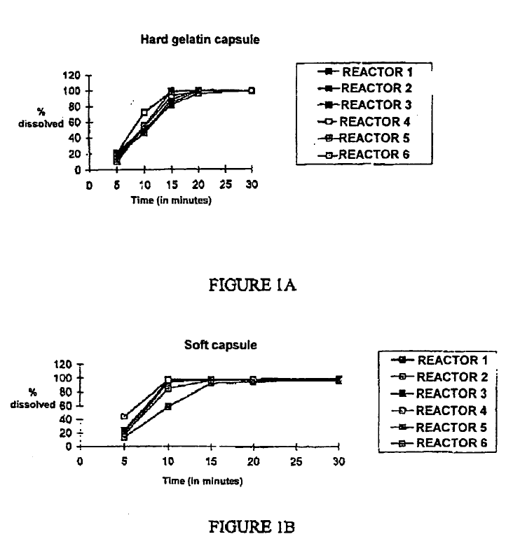

Furthermore, and unexpectedly, the dissolution rate of vinorelbine ditartrate contained in the hard gelatin capsule in example 3 above, in the dispersed state, is very similar to the dissolution dynamics of vinorelbine ditartrate contained in the dissolved state in the soft capsule. The dissolution profiles in 900 ml of water at 37° C., 50 rpm, for six samples of a batch of each galenic form, are given in FIGS. 1A and 1B appended. The process used is the rotating plate process given in the European Pharmacopoeia 2.9.3. Dissolution of vinorelbine ditartrate is 100% complete in less than 30 minutes.

Unit doses of hard gelatin capsules, expressed in basic vinorelbine, are between 5 and 100 mg and are advantageously equal to 20 mg, 30 mg, 40 mg and 80 mg.

However, this invention can be used particularly to obtain unit doses of more than 100 mg, and up to 300 mg, by injection molding.

1. A solid and stable dispersion of a hydrosoluble derivative of vinca alkaloids in at least one polyethyleneglycol with a molecular mass between 800 and 30,000.

2. The solid and stable dispersion according to claim 1, wherein the hydrosoluble derivative of vinca alkaloids is a derivative of vinorelbine.

3. The solid and stable dispersion according to claim 1 wherein the polyethyleneglycol has a molecular mass of between 1,000 and 6,000.

4. The solid and stable dispersion according to claim 1 wherein the dispersion further comprises a plastifier or a structuring agent.

5. The solid and stable dispersion according to claim 1 wherein it is in monolithic form.

6. The solid and stable dispersion according to claim 5, wherein the solid dispersion is distributed in a hard gelatin capsule.

7. The solid and stable dispersion according to claim 5, wherein the solid dispersion, associated with compression excipients, is in the form of a tablet.

8. The solid and stable dispersion according to claim 1 wherein it is in the form of divided pellets.

9. The solid and stable dispersion according to claim 8, wherein the solid dispersion is in the form of divided pellets distributed in a hard gelatin capsule.

10. The solid and stable dispersion according to claim 1 wherein the ratio of the masses of the hydrosoluble derivative of vinca alkaloids is between 1.5:1 and 1:10.

11. A process for manufacturing a stable pharmaceutical composition for oral administration, the Process comprising
heating polyethyleneglycol to a temperature slightly greater than its melting temperature to bring it to a liquid state,
mixing a hydrosoluble derivative of vinca alkaloid in powder form while stirring with polyethyleneglycol obtained in the previous step, to form a dispersions and
cooling the dispersion to bring it into a solid state, wherein the resulting composition comprises a solid and stable dispersion of the hydrosoluble derivative of vinca alkaloid in the Dolvethvleneglvcol with a molecular mass of between 800 and 30,000.

12. The process according to claim 11, wherein the polyethyleneglycol is heated in the presence of a plastifier, when a solid polyethyleneglycol is used up to a maximum temperature of 80° C.

13. The process according to claim 11, wherein a structuring agent such as silica, microcrystalline cellulose or polyethyleneglycol oxide is added to the dispersion, when a semi-solid polyethyleneglycol is used.

14. The process according to claim 11, further comprising distributing the dispersion in hard gelatin capsules.

15. The process according to claim 11 wherein the dispersion is extruded to obtain pellets to make tablets or hard gelatin capsules.

16. The process according to claim 11 wherein the dispersion is coextruded with a natural or synthetic film-forming polymer to obtain film-coated tablets.

17. The process according to claim 16, wherein the film-coated tablets are prepared in a fluidized air bed or a turbine.

18. The solid and stable dispersion according to claim 2, wherein the derivative of vinorelbine is vinorelbine ditartrate.

19. The solid and stable dispersion according to claim 2, wherein the polyethyleneglycol has a molecular mass of between 1,000 and 6,000.

20. The solid and stable dispersion according to claim 2, wherein the dispersion further comprises a plastifier or a structuring agent.

21. The solid and stable dispersion according to claim 2, wherein it is in monolithic form.

22. The solid and stable dispersion according to claim 2, wherein it is in the form of divided pellets.
